(19) United States
(12) Patent Application Publication
(10) Pub. No.: US 2009/0204124 A1
(43) Pub. Date: Aug. 13, 2009

(54) OBSTETRICAL VACUUM EXTRACTOR WITH OVER-TRACTION RELEASE

(76) Inventor: Michael G. Ross, Los Angeles, CA (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/371,263

(22) Filed: Feb. 13, 2009

Related U.S. Application Data
(60) Provisional application No. 61/028,243, filed on Feb. 13, 2008.

(51) Int. Cl. A61B 17/42

(52) U.S. Cl. 606/123

(57) ABSTRACT

A vacuum extractor for obstetrical use comprises a vacuum cup at one end of an elongated stem having a handle at the opposite end. The vacuum cup is sealed over a portion of the head of the fetus and a vacuum source, usually operating through the stem, connects to the inner side of the cup and secures it to the fetal head. A strain sensor is connected to the stem. When the force applied to the cup through the handle and stem exceeds a predetermined maximum, a valve connecting the vacuum pressure to the atmosphere is opened so as to release the vacuum pressure from the cup.

### CROSS-REFERENCE TO RELATED APPLICATIONS

This application claims priority of U.S. Provisional Patent Application Ser. No. 61/028,243 filed Feb. 13, 2008, which is incorporated herein by reference.

### FIELD OF THE INVENTION

This invention relates to an improved vacuum extractor for childbirth including a vacuum cup connected to a handle through a stem and more particularly to such an extractor including a traction gauge which measures the extraction force applied to the fetus through the cup and releases the vacuum on the cup when the extraction force exceeds a predetermined value.

### BACKGROUND OF THE INVENTION

Vacuum extractors have long been used as a tool to assist delivery, primarily in vaginal deliveries. These extractors are often used when spontaneous vaginal delivery is not possible or is unsuccessful. They typically employ a bell shaped vacuum cup connected to a handle that is used to apply a tractive force to the cup with a tube being connected by an elongated stem. A manually or electrically powered suction device is attached to the cup either through a peripherally located vacuum port or through the stem.

In use, the surgeon attaches the open end of the vacuum cup over the fetal head. A vacuum is then drawn in the cup either through use of a manually operated vacuum cup or an electrical vacuum pump. The physician then pulls on the handle to apply a delivering force to the fetal head. The force applied to the extractor must be limited to prevent harm to the fetus. With conventional vacuum extractors reliance is placed on the physician's estimate of the force being applied to avoid the imposition of excessive forces.

It has been proposed to provide vacuum extractors incorporating sensors which measure the force applied between the vacuum cup and the handle to provide the physician with an indication of when excessive forces are being applied.

U.S. Pat. No. 6,355,047 discloses an extractor incorporating such a sensor and provides a slipping mechanism which causes the length of the force center to increase when an applied traction force exceeds a predetermined level. The physician may then use his judgment as to whether to continue to apply a greater force or to terminate use of the vacuum extractor. However, in some situations excessive force has been applied despite the incorporation of a force sensor and/or a slipping mechanism, resulting in the breaking of blood vessels connecting the fetal scalp from its underlying surface.

### SUMMARY OF THE INVENTION

The present invention is accordingly directed toward an obstetrical vacuum extractor including a traction sensor connected to the air valve which releases the vacuum within the suction cup when an excessive, predetermined, force is applied to the handle of the extractor. The birthing attendant then may reattach the cup to the fetal scalp and reapply the vacuum, or resort to other means to complete the delivery. In a preferred embodiment of the invention, which will subsequently be disclosed in detail, the sensor includes a gauge operative to provide a visual indication to the user of the extraction force being exerted. The vacuum gauge may be incorporated in the conduit between the vacuum cup and the vacuum source, or alternatively it may be built into the stem, using a strain gauge or the like.

### BRIEF DESCRIPTION OF THE DRAWINGS

Other objects, advantages and applications of the present invention will be made apparent by the following detailed description of a preferred embodiment of the invention. The description makes reference to the accompanying drawings in which:

FIG. 1 is a side view of an obstetrical vacuum extractor forming a first embodiment of the invention;

FIG. 2 is a breakaway section through the stem of the extractor of FIG. 1 illustrating the pressure sensor and the relief valve controlled by the pressure sensor; and

FIG. 3 is a partial side view of a second embodiment of the invention wherein the digital pressure sensor and a relief valve controlled by the detection of excess pressure by the sensor are located in the vacuum line to the extractor body.

### DETAILED DESCRIPTION OF THE PREFERRED EMBODIMENTS

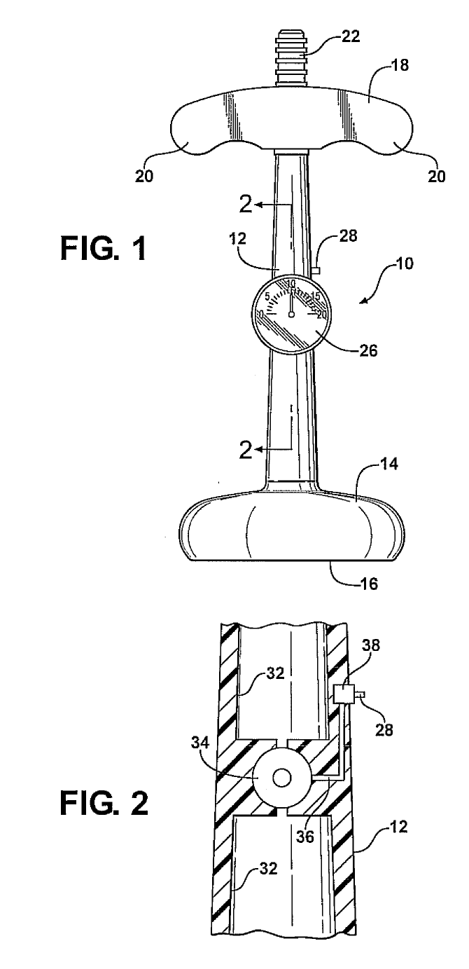

Referring to the drawings, FIG. 1 illustrates a preferred embodiment of my invention constructed on an obstetrical vacuum extractor of the general type illustrated in U.S. Pat. No. 5,019,086. The extractor, generally indicated at 10, has an elongated hollow stem 12, preferably formed of plastic. The lower end of the stem, as illustrated in FIG. 1, is connected to a resilient cup 14 which is open at its lower end 16. The cup is formed as a shell and the interior of the cup 14 communicates with the hollow section of the stem 12.

In alternate embodiments of the invention the cup 14 can be rigid and made of metal, although resilient cups are more popular with obstetricians.

The upper end of the stem 12 is connected to a rigid handle 18 consisting of two ears 20 which project on opposite sides of the connection of the handle to the stem. The upper end of the hollow within the stem is joined to a connector 22 which is adapted to receive a flexible vacuum hose (not shown). A plurality of ribs 24 formed along the length of the connector 22 provides an airtight seal for the connecting air hose.

In use, the open end 16 of the cup 14 is brought into contact with the fetal scalp and a vacuum source is joined to the connector 22. The vacuum source may be mechanically actuated with a pump powered by an assistant, or it may take the form of an electrically powered vacuum device. The suction force from the vacuum device is communicated through the stem 12 to the cup 14 and draws a vacuum which securely retains the open end 16 of the cup in contact with the fetal scalp. The physician then grasps the handle 18 and pulls on the extractor to exert a force on the fetus which assists in the delivery of the fetus through the vagina.

The vacuum extractor 10 differs from conventional vacuum extractors in two respects: First, an analog gauge 26 is positioned on the stem 12 and connected so as to sense the force applied to the fetal scalp by manipulation of the device 12. That value is exhibited on the face of the gauge 26. Secondly, a relief conduit 28 is positioned on the body of the stem so as to connect the interior of the stem, and thus the active area of the cup 16, to atmospheric pressure under certain conditions.

Referring to FIG. 2, which is a partial cross section through the extractor 10, taken along lines 2-2 of FIG. 1, the stem has an interior hollow 32 which communicates the vacuum from the connector 22 to the fetal scalp through the open end 16 of the cup 14. The vacuum drawn on the scalp passes through the gauge 34, connected to the dial 26. The gauge 34 also generates an electrical signal on output lines 36 when a predetermined value of vacuum, which represents the maximum safe extraction pressure, is exerted. The gauge is preferably battery powered. The lines 36 connect to a valve 38 which connects to the atmosphere through the conduit 28. Thus, when the pressure sensed by the gauge 34 exceeds the predetermined maximum value, the vacuum is connected directly to the atmosphere, relieving the vacuum pressure between the cup 14 and the fetal scalp.

In other embodiments of the invention the gauge 26 could present a digital output and/or the trigger value at which the gauge sends a signal to the valve 38 could be adjusted.

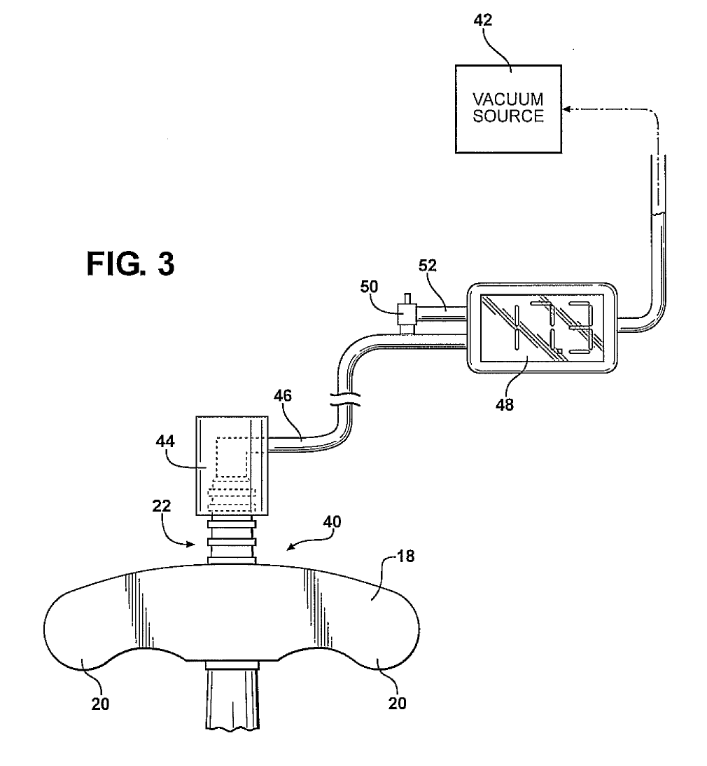

A second embodiment of the invention is illustrated in FIG. 3. The handle end of a vacuum extractor is generally indicated at 40. The vacuum extractor 40 may be identical to the vacuum extractor 10 and to the prior art in that it does not include a gauge 26 or a relief conduit 26; alternatively, in this embodiment of the invention those units are incorporated in the line connecting the vacuum extractor to the vacuum source.

As illustrated in FIG. 3, the handle 18 of the vacuum extractor 40 is equipped with a vacuum inlet port 22 of the same type as illustrated on the extractor 10 of FIGS. 1 and 2. A vacuum source 42, preferably electrically powered, draws a vacuum through a line 44 which is joined to the connector 22 by a female tubular connector 44 which slides over the male connector 22. Also connected in the line 46 is a digital gauge 48, which is preferably adjustable to a predetermined limit decided on by the attending physician, and a relief valve 50 connected to the gauge by line 52. When the limit set on the gauge 48 is reached, a signal is sent to the valve 50 which connects the line 46 to the atmosphere and thus relieves the vacuum on the cup 14 attached to the fetal scalp. The system operates in just the same way as the extractor 10 of FIG. 1, but incorporation of the sensor 48 in the valve 50 and the input line 46 allows the invention to be used with a conventional extractor which does not have its own pressure gauge and relief line.

Having thus described my invention I claim:

1. A vacuum extractor for use in an obstetrical delivery, comprising:
an elongated stem;
an open-ended cup supported on one end of the stem with the open end directed away from the stem;
a handle supported on the opposite end of the stem;
a vacuum source connected to the interior of the cup so that when the open end of the cup is brought into contact with the fetal scalp a vacuum is drawn securing the cup to the scalp;
a vacuum sensor adapted to sense the vacuum exerted between the cup and the fetal scalp;
a relief valve having one end subject to said vacuum and the other end connected to the ambient atmosphere; and
a connection between the sensor and the valve operative to open the valve, so as to relieve the vacuum securing the cup to the fetal scalp at such time as the vacuum pressure attains a predetermined value.

2. The vacuum extractor of claim 1 in which the cup has a resilient edge.

3. The vacuum extractor of claim 1 wherein the cup has a rigid edge.

4. The vacuum extractor of claim 1 further including a pressure gauge supported on the exterior of the extractor and connected to the sensor so as to provide a visual indication of the vacuum pressure retaining the cup to the fetal scalp.

5. The vacuum extractor of claim 1 in which the visual indication provided by the sensor gauge is digital.

6. A system for controlling an obstetrical vacuum extractor of the type having an elongated stem, a cup at a first end of the stem, a handle at the second end of the stem and a vacuum source for applying a vacuum to the interior of the cup while it is pressed against the scalp of a fetus so as to secure the cup and the extractor to the fetal scalp, comprising:
a vacuum sensor adapted to sense the vacuum pressure applied between the cup and the fetal scalp;
a relief valve connected at one end to the vacuum securing the cup to the fetal scalp and the other end to the atmosphere; and
an electrical connection between the sensor and the relief valve, operative to open the relief valve so as to connect the vacuum to the atmosphere at such time as the sensor detects a predetermined pressure.

7. The system of claim 6 wherein the predetermined vacuum may be adjusted.

8. The system of claim 6 in which the vacuum extractor includes a port adapted to receive a vacuum line and both the sensor and relief valve are connected to the vacuum line.

9. The system of claim 6 including a male connector and the extractor adapted to receive one end of a vacuum line slipped over the male connector; and in which the relief valve and the sensor are connected to the vacuum line.
